(19) United States
(12) Patent Application Publication
(10) Pub. No.: US 2024/0350784 A1
(43) Pub. Date: Oct. 24, 2024

(54) MICRONEEDLE PATCH AND MANUFACTURING METHOD THEREOF

(71) Applicant: DARWIN PRECISIONS CORPORATION, Hsinchu County (TW)

(72) Inventors: YunPei Yang, Hsinchu County (TW); JEN SHUN LIN, Hsinchu County (TW)

(21) Appl. No.: 18/369,851

(22) Filed: Sep. 19, 2023

(30) Foreign Application Priority Data
Apr. 20, 2023 (TW) 112114729

(51) Int. Cl. A61M 37/00

(52) U.S. Cl. CPC A61M 37/0015; A61M 2037/0023; A61M 2037/0053

(57) ABSTRACT

A microneedle patch includes a microneedle structural layer, an adhesive layer, and a carrier layer. The adhesive layer is disposed between the microneedle structural layer and the carrier layer, and is connected to the microneedle structural layer and the carrier layer. The microneedle structural layer comprises a base layer and a plurality of microneedles, and the base layer has a first side and a second side opposite to the first side, and the first side faces the adhesive layer. The plurality of microneedles are spaced apart from each other and disposed on the second side, and include a plurality of first microneedles and at least one second microneedle. The at least one second microneedle is located at an edge of the microneedle structural layer, and the height of the at least one second microneedle is less than that of each first microneedle.

### FIELD OF THE INVENTION

The present invention relates to the field of transdermal drug delivery, in particular to a microneedle patch.

### BACKGROUND OF THE INVENTION

The microneedle patch (MNP) is a new type of transdermal drug delivery system (TDDS) that combines the advantages of patch and subcutaneous injection. As the microneedles on the patch are too short to touch the nerves, not only does not cause the pain of subcutaneous injection, but also can easily carry macromolecular medicine through the stratum corneum into the human body. Since the MNP technology has been promoted in 1998, there has been a vigorous development in aesthetic medicine, medicine, vaccines and measurement systems, and aesthetic medicine products are now commercially available, while the product development in the other three fields has gradually been commercialized.

In the manufacturing of MNP, different metal molds are usually prepared according to the product design, so a certain cost is required; however, a female die made of the conventional metal mold has a problem of misalignment with the release mold in the manufacturing process due to the material properties thereof, thereby producing non-performing products. It can be seen that the area of the microneedle on the patch deviates from the correct position in non-performing products, affecting both the user experience and microneedle administration.

### SUMMARY OF THE INVENTION

The present invention provides a microneedle patch having a good positional accuracy of a microneedle area.

The present invention also provides a method for manufacturing a microneedle patch, which can improve the yield rate of the microneedle patch production and ensure that the location of the microneedle area conforms to the design.

The present invention also provides a metal mold that can be used to improve the yield rate of the microneedle patch production and the positional accuracy of the microneedle area.

The microneedle patch according to the present invention comprises a microneedle structural layer, an adhesive layer, and a carrier layer. The adhesive layer is disposed between the microneedle structural layer and the carrier layer, and is connected to the microneedle structural layer and the carrier layer. The microneedle structural layer comprises a base layer and a plurality of microneedles, and the base layer has a first side and a second side opposite to the first side, and the first side faces the adhesive layer. The plurality of microneedles are spaced apart from each other and disposed on the second side, and include a plurality of first microneedles and at least one second microneedle. The at least one second microneedle is located at an edge of the microneedle structural layer, and a height of the at least one second microneedle is less than a height of each of the first microneedles.

The method for manufacturing the microneedle patch according to the present invention comprises steps of: providing a male die, and the provided male die has a bearing seat and a plurality of microneedle structures, and the plurality of microneedle structures are disposed on the bearing seat and spaced apart from each other; dispensing a polymeric material into the male die and forming a female die; dispensing a biocompatible material into the female die and forming a microneedle structural layer, the microneedle structural layer comprises a plurality of microneedles configured to be spaced apart from each other; disposing at least one release film and at least one carrier layer on a side opposite to a side of the microneedle structural layer where a plurality of microneedles are provided, the at least one release film has a plurality of hollow areas, and each of the hollow areas has a specific shape, and the at least one carrier layer is disposed on the at least one release film and covers the plurality of hollow areas; separating the microneedle structural layer and the female die; and cutting the microneedle structural layer according to the specific shape of each hollow area.

The metal mold according to the present invention is used for manufacturing a microneedle patch, and comprises a bearing seat and a plurality of microneedle structures, the bearing seat has a single area thereon, and the plurality of microneedle structures are disposed in the single area at a distributed density.

According to the present invention, since a metal mold is used, and a plurality of microneedle structures of the metal mold are disposed in a single area of a bearing seat, alignment errors of a female die can be avoided in the manufacturing process, thereby contributing to improvement in the yield rate of microneedle patch production and ensuring that the location of the microneedle area conforms to the design. The microneedle area of the present invention helps to enhance the user's experience and ensure that the microneedle is administered at the correct site due to good positional accuracy.

Other objectives, features and advantages of the invention will be further understood from the further technological features disclosed by the embodiments of the invention wherein there are shown and described preferred embodiments of this invention, simply by way of illustration of modes best suited to carry out the invention.

### BRIEF DESCRIPTION OF THE DRAWINGS

FIG. 1 is a schematic cross-sectional diagram of a microneedle patch according to a first embodiment of the present invention.

FIG. 2 is a schematic cross-sectional diagram of a microneedle patch according to a second embodiment of the present invention.

FIG. 3 is a schematic cross-sectional diagram of a microneedle patch according to a third embodiment of the present invention.

FIG. 4 is a schematic cross-sectional diagram of a microneedle patch according to a fourth embodiment of the present invention.

FIG. 5 is a flow chart illustrating a method for manufacturing a microneedle patch according to an embodiment of the present invention.

FIGS. 6A-6C are schematic diagrams illustrating steps of a method for manufacturing a microneedle patch according to the first embodiment of the present invention.

FIG. 7 is a schematic top view showing the positional relationship between a release film and a microneedle structural layer according to an embodiment of the present invention.

FIG. 8 is a schematic top view showing the positional relationship between a release film and a microneedle structural layer according to another embodiment of the present invention.

FIGS. 9A-9C are schematic photomicrographs of microneedles according to embodiments of the present invention.

### DETAILED DESCRIPTION OF PREFERRED EMBODIMENTS

The foregoing and other technical contents and other features and advantages of the present invention will be clearly presented from the following detailed description of a preferred embodiment in cooperation with the accompanying drawings. Directional terms mentioned in the following examples, are only used to describe directions referring to the attached drawings. Therefore, the directional terms used are for illustration and not for limitation. In addition, terms such as “first” and “second” mentioned in this specification or the claims are only used to name elements or to distinguish different embodiments or scopes, and are not intended to limit the upper or lower limit on the number of elements.

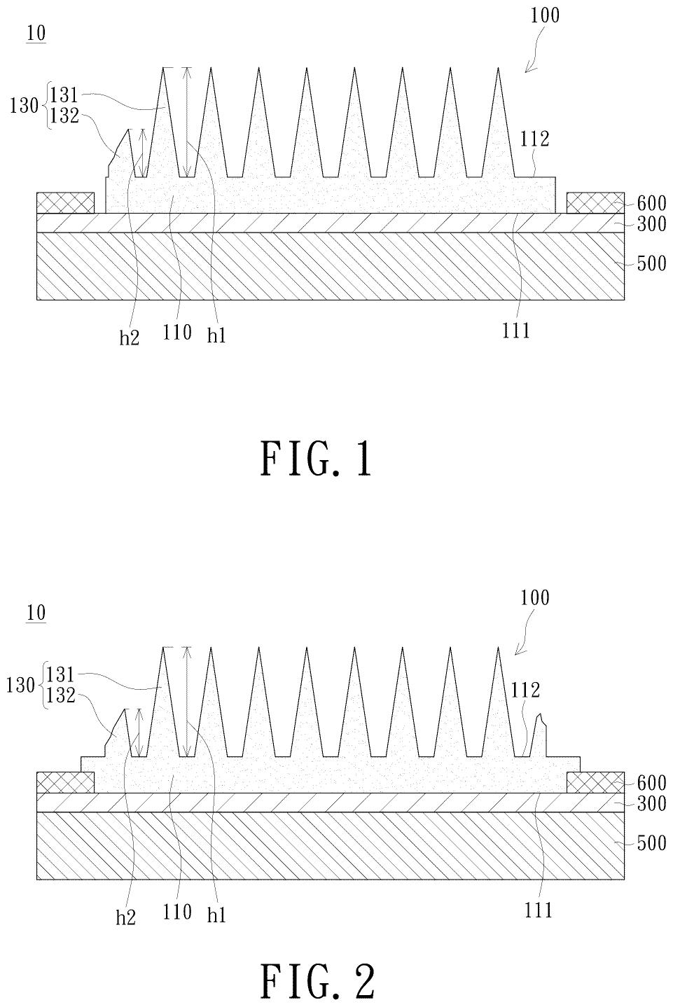

FIG. 1 is a schematic cross-sectional diagram of a microneedle patch according to an embodiment of the present invention. As shown in FIG. 1, a microneedle patch 10 comprises a microneedle structural layer 100, an adhesive layer 300, and a carrier layer 500. The adhesive layer 300 is disposed between the microneedle structural layer 100 and the carrier layer 500, and is connected to the microneedle structural layer 100 and the carrier layer 500. The microneedle structural layer 100 comprises a base layer 110 and a plurality of microneedles 130, and the base layer 110 has a first side 111 and a second side 112 opposite to the first side 111. The first side 111 faces the adhesive layer 300, and the second side 112 is provided with a plurality of microneedles 130. The plurality of microneedles 130 are spaced apart from each other and disposed on the second side 112 at an appropriate distribution density. In some embodiments of the present invention, the plurality of microneedles 130 are uniformly distributed across the second side 112 with substantially equal spacing between adjacent microneedles 130. The distance between adjacent microneedles 130 can be, for example, 0.5 to 5 mm.

The material of the carrier layer 500 can be a synthetic or natural fabric, such as a woven, non-woven or knitted fabric, a synthetic or natural polymer. The carrier layer 500 is preferably compliant and flexible to facilitate even attachment to a biological surface. For example, the carrier layer 500 can be, for example, a film or a cloth. The material of the carrier layer 500 can also be selected on the basis of a plurality of functional requirements such as water resistance, air permeability, bacteriostasis, odor, aesthetics, feel, but also with reference to commercially available medical consumables such as bandages, tapes, and patches.

Preferably, the plurality of microneedles 130 of the microneedle structural layer 100 are integrally formed with the base layer 110, and in a preferred embodiment of the present invention, the material of the microneedle structural layer 100 is a biocompatible material and/or a biodegradable material. Further, the material of the microneedle structural layer 100 is preferably a high molecular material having solubility and swelling properties, and can be exemplified by, but not limited to, maltose, sucrose, trehalose, lactose, dextrin, maltodextrin, beta-cyclodextrin, 2-hydroxypropyl-beta-cyclodextrin, dextran, amylopectin, sodium hyaluronate, methyl vinyl ether-maleic anhydride copolymer, sodium carboxymethyl cellulose, methyl cellulose, hydroxypropyl methyl cellulose, hydroxypropyl cellulose, gelatin, polyvinyl alcohol, polyvinyl pyrrolidone, polyethylene glycol, polylactic acid, polyglycolic acid, polylactic acid-glycolic acid copolymer, chitosan, and combinations thereof. In addition, the microneedle structural layer 100 may contain pharmacologically active molecules such as transdermal drugs.

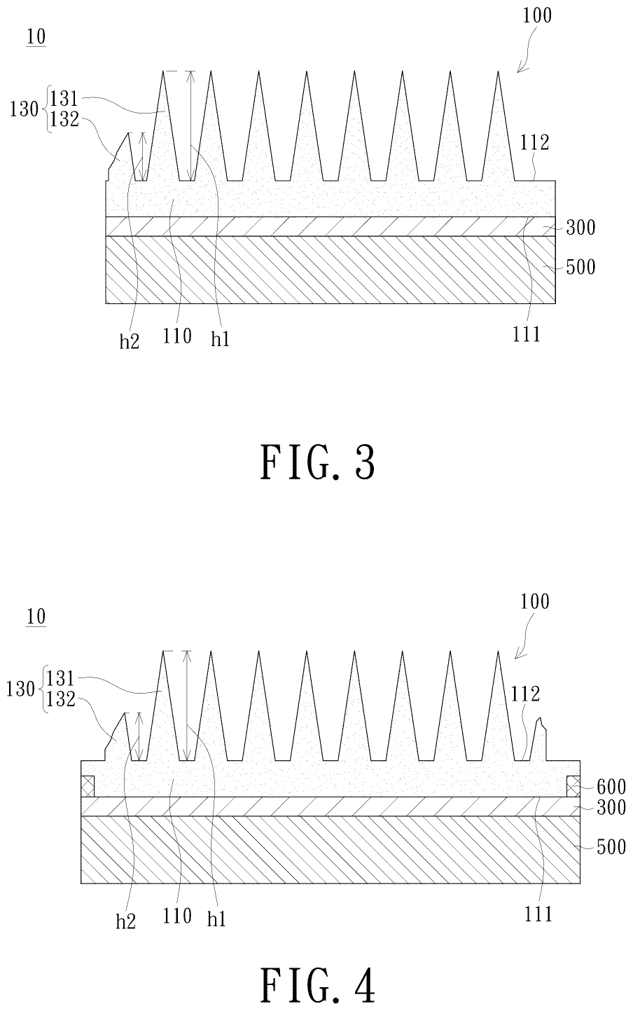

In the present embodiment, the microneedle 130 further includes a first microneedle 131 and at least one second microneedle 132, and the second microneedle 132 is located at an edge of the microneedle structural layer 100. There are a plurality of first microneedles 131 having the same shape, e.g., a cone, a triangular cone, or a quadrangular cone, and having approximately the same height as each other, e.g. 150 to 1000 μm. The shape of the second microneedle 132 is different from the shape of the plurality of first microneedles 131, and a height h2 of the second microneedle 132 is generally smaller than a height h1 of the first microneedles 131, and the second microneedle 132 is a deformation structure of the first microneedles 131. In the embodiment of the present invention, the second microneedle 132 is formed from the first microneedle 131 through a cutting step (described later). As shown in FIG. 2, the embodiment of the present invention can further comprise a plurality of second microneedles 132 having different shapes.

As shown in FIGS. 1 and 2, the microneedle patch 10 can further comprise a release film 600. The release film 600 is located at the periphery of the carrier layer 500 and is disposed at least partially along the periphery of the carrier layer 500 on the carrier layer 500. In the embodiment shown in FIG. 1 or FIG. 2, the release film 600 can be considered to be disposed entirely on the carrier layer 500 along the periphery of the carrier layer 500, although in other embodiments, the release film 600 can also partially protrude outside the carrier layer 500.

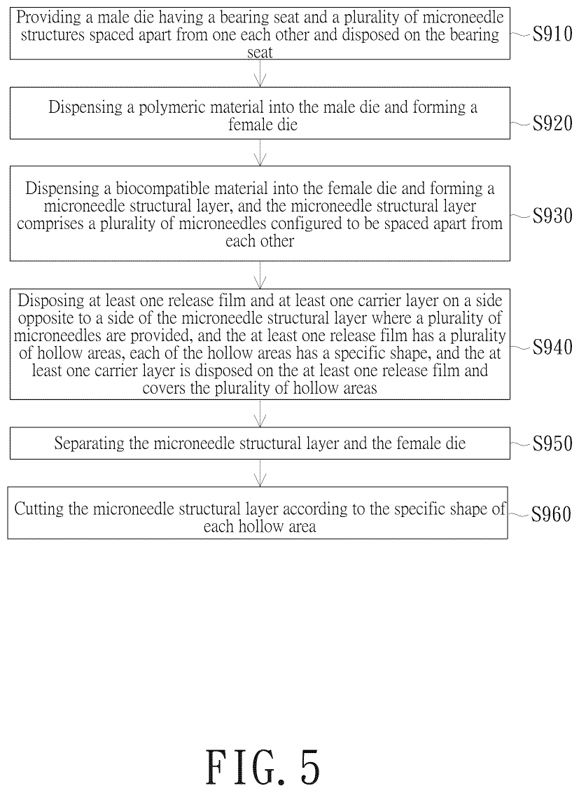

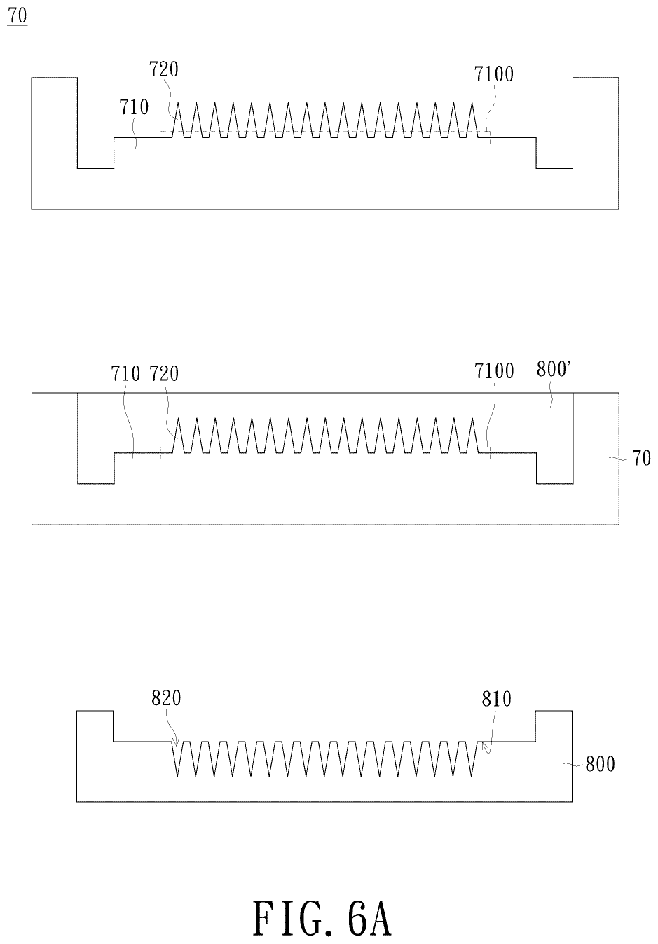

The release film 600 can also be connected to the carrier layer 500 through the adhesive layer 300. In some embodiments, the microneedle structural layer 100 and the carrier layer 500 are similar in shape, for example, circular, but the present invention is not limited thereto. For example, the carrier layer 500 has a rectangular shape and the microneedle structural layer 100 can have an elliptical shape, or the carrier layer 500 has a square shape and the microneedle structural layer 100 can have a circular shape. In a preferred embodiment of the present invention, as shown in FIGS. 1 and 2, the carrier layer 500 is larger than the microneedle structural layer 100, and the release film 600 surrounds the microneedle structural layer 100. However, in other embodiments, as shown in FIGS. 3 and 4, the size of the carrier layer 500 and the size of the microneedle structural layer 100 can be substantially the same.

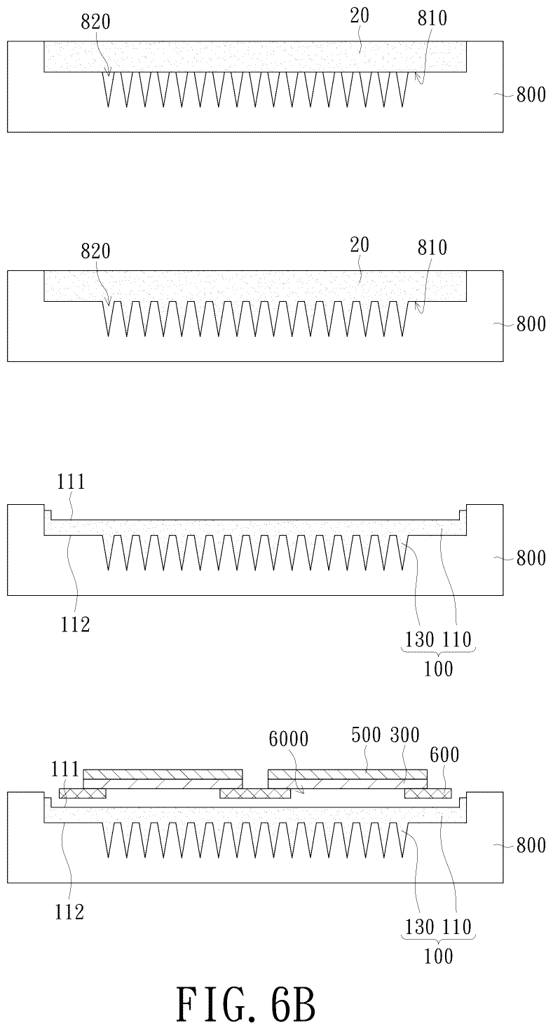

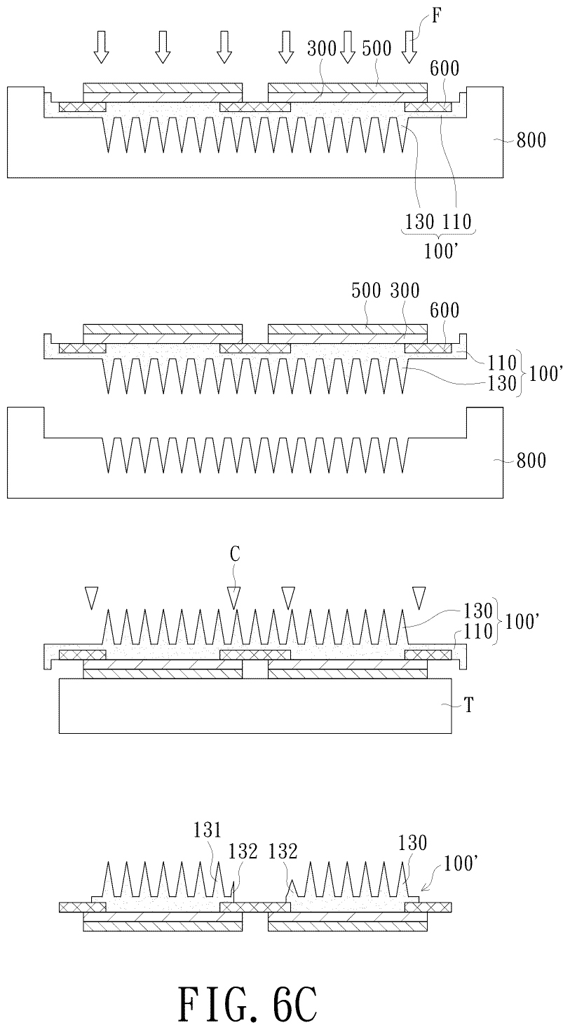

The present invention also provides a method for manufacturing a microneedle patch, as shown in FIG. 5, including steps S910-S960. Step S910 comprises: providing a male die having a bearing seat and a plurality of microneedle structures spaced apart from each other and disposed on the bearing seat. Step S920 comprises: dispensing a polymeric material into the male die and forming a female die. Step S930 comprises: dispensing a biocompatible material into the female die and forming a microneedle structural layer, and the microneedle structural layer comprises a plurality of microneedles configured to be spaced apart from each other. Step S940 comprises: disposing at least one release film and at least one carrier layer on a side opposite to a side of the microneedle structural layer where a plurality of microneedles are provided, and the at least one release film has a plurality of hollow areas, each of the hollow areas has a specific shape, and the at least one carrier layer is disposed on the at least one release film and covers the plurality of hollow areas. Step S950 comprises: separating the microneedle structural layer and the female die. Step S960 comprises: cutting the microneedle structural layer according to the specific shape of each hollow area. FIGS. 6A-6C are schematic illustrations of the implementation of steps S910-S960.

The male die 70 of step S910 is a metal mold. In embodiments of the present invention, the material of the male die 70 is not limited and can be, for example, titanium, copper, aluminum, nickel, tungsten, stainless steel, titanium alloys, nickel alloys, aluminum alloys, copper alloys. In a preferred embodiment of the present invention, as shown in FIG. 6A, the male die 70 further has a single area 7100 on the bearing seat 710. A plurality of microneedle structures 720 are spaced apart from each other and disposed in the single area 7100 at an appropriate distribution density. In some embodiments of the present invention, the plurality of microneedle structures 720 are uniformly distributed across the single area 7100 with substantially equal spacing between adjacent microneedle structures 720. The distance between adjacent microneedle structures 720 can be, for example, between 0.5 and 5 mm. The plurality of microneedle structures 720 have the same shape, e.g., a cone, a triangular cone, or a quadrangular cone, and have approximately the same height as each other, e.g. 150 to 1000 μm.

The polymeric material 800′ of step S920 can be, for example, polyethylene, polypropylene, polylactic acid, polybutylene succinate, and polydimethylsiloxane. As shown in FIG. 6A, step S920 further comprises dispensing the polymeric material 800′ into the single area 7000 and the plurality of microneedle structures 720 thereon, such that a plurality of tapered holes 820 are formed on the female die 800. A shape of the tapered hole 810 corresponds to a shape of the microneedle structure 720. In a preferred embodiment of the present invention, the female die 800 can further have a bottom surface area 810 therein that corresponds to the single area 7100 of the male die 70. The plurality of the tapered holes 820 are spaced apart from each other and disposed at an appropriate distribution density in the bottom surface area 810.

The biocompatible material 20 of step S930 is as previously described. In some embodiments of the present invention, step S930 can further comprise adding a pharmacologically active component, such as a transdermal medicine, to the biocompatible material 20 to render the microneedle structural layer 100′ pharmacologically active. As shown in FIG. 6B, step S930 further comprises dispensing the biocompatible material 20 into the female die 800 and the bottom surface area 810 thereof, and the plurality of tapered holes 820 in the bottom surface area 810 so that the microneedle structural layer 100′ has the base layer 110 and the plurality of microneedles 130. Step S930 further comprises letting the base layer 110 have the first side 111 and the second side 112 opposite to the first side 111. The second side 112 is formed by dispensing the biocompatible material 20 into the bottom surface area 810. The plurality of microneedles 130 are spaced apart from each other and configured on the second side 112 at an appropriate distribution density. The microneedles 130 can be used to deliver medicine transdermally to an organism. In some embodiments of the present invention, the microneedles 130 contain medicine. When the microneedles 130 penetrate the stratum corneum, the polymeric material of the microneedles 130 swells or dissolves in the skin and allows the medicine to be gradually released and diffused into the systemic blood vessels for efficacy.

Step S930 can further comprise letting the biocompatible material 20 dry to form the microneedle structural layer 100′. As shown in FIGS. 6B-6C, step S940 further comprises disposing at least one release film 600 and at least one carrier layer 500 on the first side 111 of the base layer 110. The at least one release film 600 and the at least one carrier layer 500 can be sequentially disposed on the first side 111, or can be combined and then disposed on the first side 111. Step S940 can further comprise disposing an adhesive layer 300 between the at least one carrier layer 500 and the microneedle structural layer 100′ and connecting the at least one carrier layer 500 and the microneedle structural layer 100′. In some embodiments of the present invention, the carrier layer 500 can be provided with the adhesive layer 300 and the release film 600 is combined, and then the combination comprising the carrier layer 500, the adhesive layer 300 and the release film 600 is disposed on the first side 111, and the adhesive layer 300 can be further bonded to the microneedle structural layer 100′.

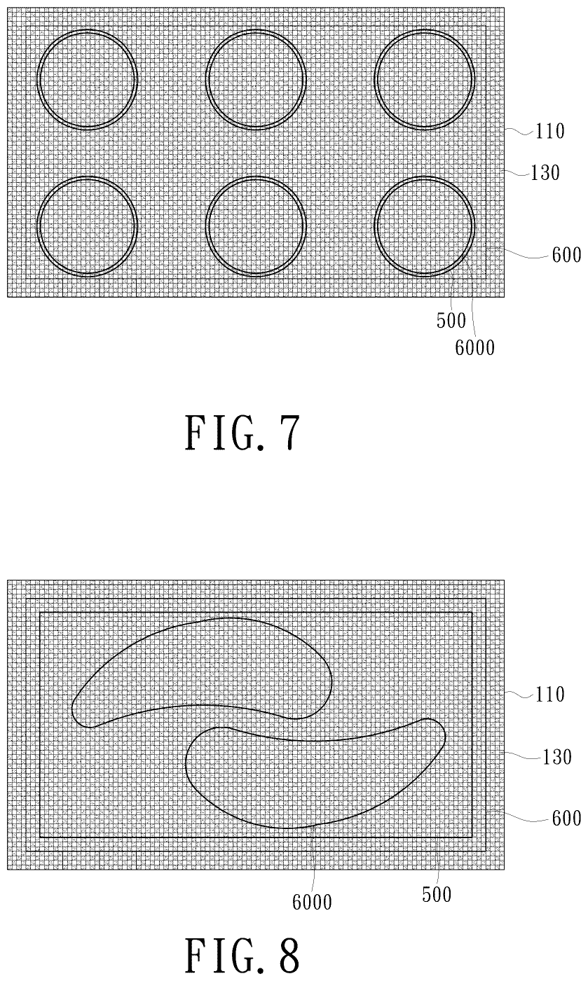

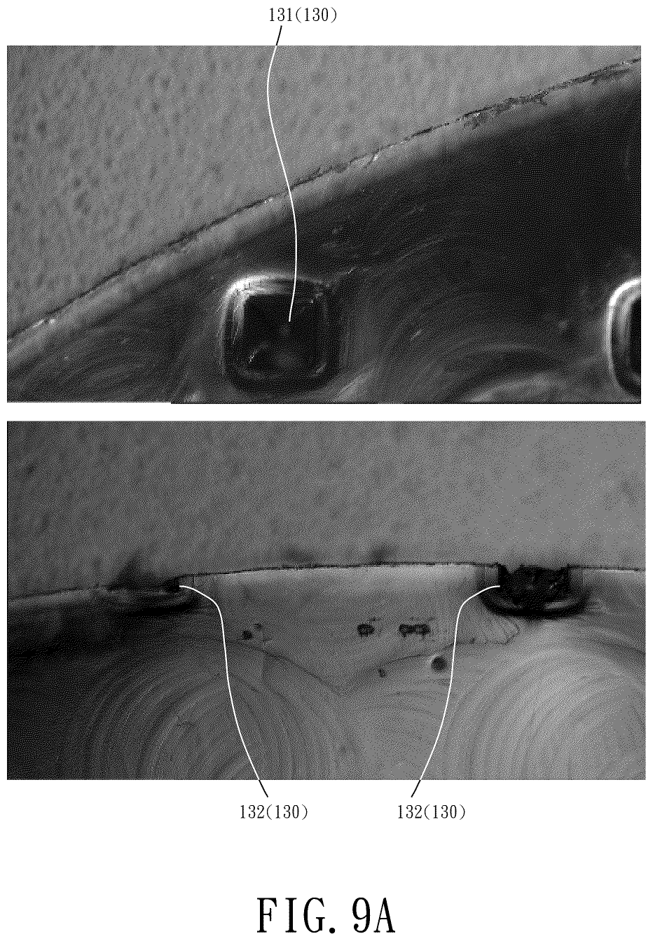

In an embodiment of the present invention, as shown in FIG. 7, one release film 600 is used in step S940 and there are a plurality of carrier layers 500. FIG. 7 shows the positional relationship between the release film 600 and the microneedle structural layer 100. As shown in FIG. 7, the release film 600 has a plurality of hollow areas 6000, and each of the hollow areas 6000 has a specific shape. For example, as shown in FIG. 7, the hollow area 6000 is circular. However, in other embodiments, as shown in FIG. 8, the hollow area 6000 is ear-shaped. In addition, the shapes of the plurality of hollow areas 6000 can be different. The specific shape of the hollow area 6000 can reflect the arrangement shape of the microneedles 130 of the manufactured microneedle patch 10 (hereinafter also referred to as a microneedle area). In addition, the number of hollow areas 6000 can be the maximum number of microneedle patches 10 that can be manufactured at one time in steps S940 to S960. For example, as shown in FIG. 7, steps S940-S960 can have six microneedle patches 10 produced at one time, while there will be two microneedle patches 10 in the embodiments shown in FIG. 8, which however are not limited thereto. As shown in FIGS. 7 and 8, the hollow area 6000 preferably comprises the area where the microneedles 130 are distributed. In the embodiment of the present invention, since the base layer 110 of the microneedle structural layer 100′ is filled with the microneedles 130, in principle there is no need in step S940 to perform alignment of the release film 600 and the hollow areas 6000 thereof with the microneedles 130, thereby also avoiding alignment errors between the hollow areas 6000 and the microneedle areas due to, for example, shrinkage or expansion of the female die 800.

As shown in FIG. 7, there can be six carrier layers 500, corresponding to six hollow areas 6000, respectively. The carrier layer 500 can further correspond to the first side 111 of the base layer 110 bounded by the hollow area 6000. However, in other embodiments, there can be one carrier layer 500, and the carrier layer 500 can be such as a rectangular one-piece structure like the release film 600. The one-piece carrier layer 500 can then be cut to separate a plurality of microneedle patches 10.

Step S940 can further comprise applying a force F to the carrier layer 500 toward the microneedle structural layer 100′ to facilitate connecting of the carrier layer 500 to the microneedle structural layer 100′. Step S950 further comprises removing the microneedle structural layer 100′ connected to the carrier layer 500 from the female die 800. Step S960 further comprises cutting the microneedle structural layer 100′ by using a cutting tool C such as a die cutting punch and the laser, to divide the microneedle structural layer 100′ into a plurality of individual microneedle structural layers 100. In the embodiment of the present invention, since the base layer 110 of the microneedle structural layer 100′ is filled with the microneedles 130, the cutting tool passing through the microneedles 130 will deform the microneedles 130 easily. The microneedle 130 formed in step S930 is also referred to the first microneedle 131 herein, and the microneedle 130 deformed by cutting in step S960 is referred to the second microneedle 132. The second microneedle 132 is generally located at the edges of the microneedle structural layer 100.

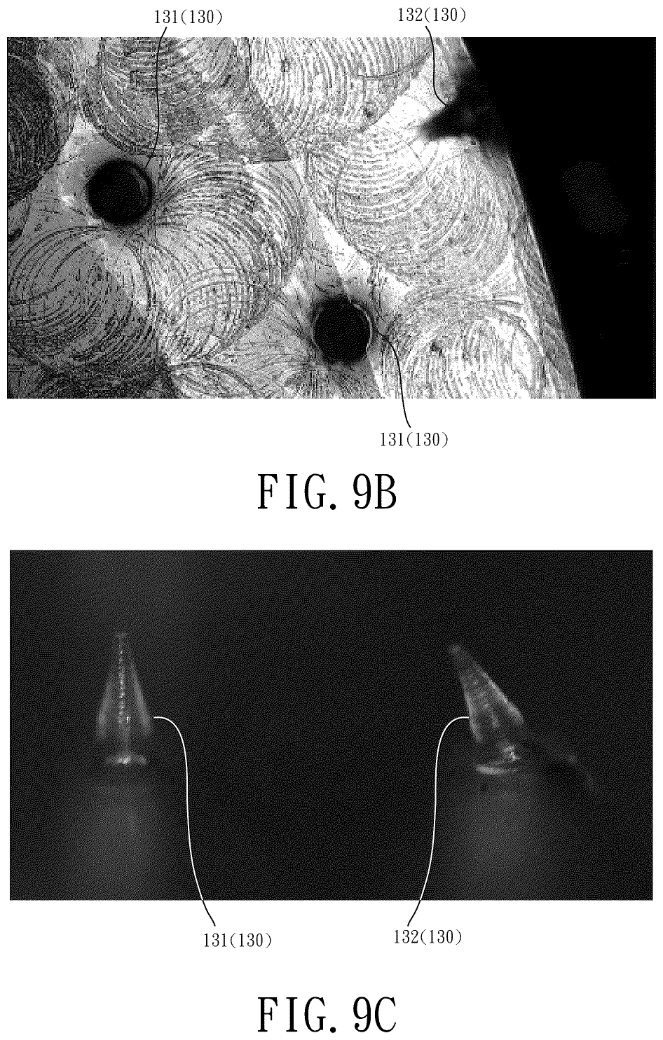

FIGS. 9A-9C illustrate various aspects of microneedle deformation. FIG. 9A is a schematic photomicrograph of microneedles according to an embodiment of the present invention, where the shape of the microneedle 130 is a quadrangular cone. As shown in FIG. 9A, the normal first microneedle 131 has a quadrangular bottom surface, and the deformed second microneedle 132 is located at the edge and loses the integrity of the bottom surface. FIG. 9B is a photomicrograph of microneedles according to another embodiment of the present invention, where the microneedle 130 is shaped as a cone and the deformed second microneedle 132 is located at the edge and loses the integrity of the bottom surface. As shown in FIG. 9C, the normal first microneedle 131 stands upright, and the second microneedle 132 is bent and deformed.

For the finished product obtained through steps S910 to S960, as shown in FIG. 6C, the release film 600 is disposed along the periphery of each carrier layer 500 and can partially protrude outside each carrier layer 500. In some embodiments of the present invention, the method for manufacturing the microneedle patch can further comprise cutting the release film to form the plurality of microneedle patches and/or to modify the release film. For example, the release film 600 can be further cut after step S960, thereby dividing the finished product into the plurality of individual microneedle patches 10, and/or cutting off portions of the release film 600 protruding outside each carrier layer 500.

Referring again to FIG. 7. As shown in FIG. 7, the shape of the carrier layer 500 is approximately the same as the shape of the microneedle area and has a circular shape, but is not limited thereto. The shape of the carrier layer 500 can also be different from the microneedle area. In some embodiments of the present invention, the method for manufacturing the microneedle patch can further comprise cutting the carrier layer, for example, cutting the one-piece carrier layer 500 according to the hollow area 6000 or the microneedle area, to divide the carrier layer 500 into plurality, and the plurality of carrier layers 500 can constitute the plurality of microneedle patches 10 respectively with the separated microneedle structural layers 100. The carrier layer 500 can be cut to have the same shape as or a different shape from the hollow area 6000 or the microneedle area, or the same size as or a different size from the microneedle structural layer 100. Referring again to FIG. 8, for example, the embodiment of FIG. 8 can be followed by cutting the release film 600 and the carrier layer 500 to form two microneedle patches 10 having ear-shaped microneedle areas, where the release film 600 and the carrier layer 500 are not limited to be ear-shaped. However, the embodiment of FIG. 8 can be formed as a single microneedle patch 10 having two ear-shaped microneedle areas.

According to an embodiment of the present invention, since the microneedle structures 720 of the male die 80 are fully disposed in the single area 7100 of the bearing seat 710 and can be used to manufacture the plurality of microneedle patches 10, a number of the microneedle structures 720 is therefore preferably greater than a sum of the number of microneedles 130 of the plurality of manufactured microneedle patches 10. According to the embodiment of the present invention, since the microneedle structures 720 of the male die 80 are fully disposed in the single area 7100 of the bearing seat 710, alignment errors between the hollow area 6000 and the microneedle area due to, for example, shrinkage or expansion of the female die 800 can be avoided in the subsequent steps, thereby contributing to improving microneedle patch production yield rate and ensuring that the location of the microneedle area conforms to the design.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A microneedle patch, comprising a microneedle structural layer, an adhesive layer, and a carrier layer, wherein
the adhesive layer is disposed between the microneedle structural layer and the carrier layer, and is connected to the microneedle structural layer and the carrier layer;
the microneedle structural layer includes a base layer and a plurality of microneedles; the base layer has a first side and a second side opposite to the first side; the first side faces the adhesive layer, the plurality of microneedles are spaced apart from each other and disposed on the second side, and comprise a plurality of first microneedles and at least one second microneedle;
wherein the at least one second microneedle is located at an edge of the microneedle structural layer, and a height of the at least one second microneedle is less than a height of each first microneedle.

2. The microneedle patch according to claim 1, wherein the plurality of first microneedles have the same height in a range of 150 to 1000 μm.

3. The microneedle patch according to claim 1, wherein a distance between adjacent microneedles of the plurality of microneedles is 0.5 to 5 mm.

4. The microneedle patch according to claim 1, wherein a shape of the at least one second microneedle is different from a shape of the plurality of first microneedles.

5. The microneedle patch according to claim 4, further comprising a plurality of second microneedles having different shapes.

6. The microneedle patch according to claim 1, further comprising a release film, the release film being disposed at the periphery of the carrier layer and at least partially along the periphery of the carrier layer.

7. The microneedle patch according to claim 6, wherein the microneedle structural layer has a shape similar to a shape of the carrier layer; the carrier layer is equal to or larger than the microneedle structural layer, and the release film surrounds the microneedle structural layer.

8. A method for manufacturing a microneedle patch, comprising:
providing a male die, the male die having a bearing seat and a plurality of microneedle structures, wherein the plurality of microneedle structures are spaced apart from each other and disposed on the bearing seat;
dispensing a polymeric material into the male die and forming a female die;
dispensing a biocompatible material into the female die and forming a microneedle structural layer, the microneedle structural layer comprising a plurality of microneedles configured to be spaced apart from each other;
disposing at least one release film and at least one carrier layer on a side opposite to a side of the microneedle structural layer where a plurality of microneedles are provided, wherein the at least one release film has a plurality of hollow areas, and each of the hollow areas has a specific shape; the at least one carrier layer is disposed on the at least one release film and covers the plurality of hollow areas;
separating the microneedle structural layer and the female die; and
cutting the microneedle structural layer according to the specific shape of each hollow area.

9. The method for manufacturing the microneedle patch according to claim 8, wherein the step of forming the female die further comprises forming a plurality of tapered holes; the plurality of tapered holes are spaced apart from each other, and a shape of the tapered holes corresponds to a shape of the microneedle structures.

10. The method for manufacturing the microneedle patch according to claim 8, wherein the step of forming the microneedle structural layer further comprises forming a base layer and a plurality of first microneedles; the base layer has a first side and a second side opposite to the first side, and the plurality of first microneedles are spaced apart from each other and disposed on the second side.

11. The method for manufacturing the microneedle patch according to claim 10, wherein the step of disposing the at least one release film and the at least one carrier layer further comprises disposing the at least one release film and the at least one carrier layer on the first side of the base layer, and disposing an adhesive layer between the at least one carrier layer and the microneedle structural layer and connecting the at least one carrier layer and the microneedle structural layer.

12. The method for manufacturing the microneedle patch according to claim 8, wherein the step of cutting the microneedle structural layer further comprises passing a cutting tool through at least one of the microneedles and deforming the at least one microneedle into at least one second microneedle.

13. The method for manufacturing the microneedle patch according to claim 8, wherein the male die further has a single area on the bearing seat, and the plurality of microneedle structures are disposed in the single area at a distribution density; the method for manufacturing the microneedle patch further comprises forming a plurality of microneedle patches, and a number of the microneedle patches is the same as a number of the hollow areas.

14. A metal mold, wherein the metal mold is used for manufacturing a microneedle patch and comprises a bearing seat and a plurality of microneedle structures, wherein:
the bearing seat has a single area thereon, and the plurality of microneedle structures are disposed in the single area at a distribution density.

15. The metal mold according to claim 14, wherein the metal mold is adapted to manufacture a plurality of microneedle patches, each of the microneedle patches respectively has a microneedle structural layer comprising a base layer and a plurality of microneedles; the plurality of microneedles are spaced apart from each other at the distribution density and disposed on the base layer, and a sum of the number of the microneedles of the plurality of microneedle patches is smaller than a sum of the number of the microneedle structures of the metal mold.
